(19) United States
(12) Patent Application Publication
(10) Pub. No.: US 2026/0151274 A1
(43) Pub. Date: Jun. 4, 2026

(54) APPENDAGE TIP BANDAGE

(71) Applicants: William R. MARTIN, Durham, NC (US); Jay A. BERKOWITZ, Penn Valley, PA (US)

(72) Inventors: William R. MARTIN, Durham, NC (US); Jay A. BERKOWITZ, Penn Valley, PA (US)

(21) Appl. No.: 19/457,675

(22) Filed: Jan. 23, 2026

Related U.S. Application Data
(63) Continuation-in-part of application No. 29/951,585, filed on Jul. 10, 2024, now Pat. No. D1119319.

(51) Int. Cl. A61F 13/10; A61F 13/00; A61F 13/06

(52) U.S. Cl. CPC A61F 13/105; A61F 13/068; A61F 2013/00093

(57) ABSTRACT

A bandage that firmly secures to an appendage (e.g., finger/toe) having a wound on or near the tip. The bandage includes first portion of material, having an absorbent layer secured to a first side, and a second portion of material, also having an absorbent layer secured to a first side and joining the first and second portions along edges of upper regions of those portions, the first sides facing each other, forming an appendage tip receptacle (ATR). Wings are provided on the first portion, one wing on each side of a lower portion of the first portion. An adhesive region is included on each wing and facing the second portion. Once the appendage tip with the wound is inserted into the ATR, the wings are wrapped around the appendage in opposite directions to overlap and pressure is applied to the overlapping wings to releasably secure the bandage to the appendage.

### CROSS-REFERENCE TO RELATED APPLICATIONS

This Continuation-in-Part application claims the benefit under 35 U.S.C. § 120 of application Ser. No. 29/951,585 filed on Jul. 10, 2024 entitled FINGER COT and whose entire disclosure is incorporated by reference herein.

### BACKGROUND OF THE INVENTION

The present invention relates to bandages, and more particularly, bandages that can be applied securely to the outer end of a body appendage such as a finger or toe to cover a wound, scrape or abrasion.

For minor cuts, scrapes or wounds or abrasions on a person's skin, an adhesive bandage is typically applied. These well-known adhesive bandages (e.g., BAND-AID® adhesive bandages) typically comprise a backing material, an adhesive layer, an absorbent pad and a release liner(s). The backing material typically comprises a plastic, fabric and or latex-free synthetic material which provides flexibility and durability and often is perforated to allow for “breathability.” The adhesive layer is a typically a pressure-sensitive adhesive that sticks to the skin and is designed to be gentle to the skin yet provide secure attachment thereto; some adhesive layers are also hypoallergenic to reduce skin irritation. The absorbent pad (also referred to as a “wound pad”) is located in the center of the bandage and typically comprises cotton or other non-stick material; this absorbent pad is purposed to absorb the exudate while protecting the wound. In many instances, the absorbent pad is coated with a non-adherent layer to prevent sticking to the wound. Finally, the protective paper or plastic layer is applied over the adhesive pad to maintain the adhesive nature of the pad until the adhesive bandage is to be used. There are typically two release liners, each one covering a respective adhesive pad on either side of the absorbent pad. Additional features may also be used, e.g., an antiseptic or antibiotic coating (e.g., bacitracin) and/or a waterproof or water-resistant backing.

These type of adhesive bandages are typically easy to apply, and secure, to relatively flat surfaces of the skin. However, when wounds, scrapes or abrasions occur to an end of an appendage, e.g., a finger or toe, etc., these adhesive bandages are not as effective. In particular, the adhesive bandage can be wound around the appendage in order to cover wound but typically cannot cover the entire wound if the wound extends on a curved surface of the appendage; alternatively, if the adhesive bandage is applied along the curved end of the appendage, it tends to disengage since the adhesive pads are located on different appendage planes.

Alternative style bandages have been developed to attempt to address such end of appendage wounds, and are frequently referred to as “finger cots.” For example, see U.S. Pat. No. 1,231,194 (Prince); 2,440,235 (Solomon); 2,243,422 (Hollander, et al.); 2,461,872 (Beatty); 2,847,005 (Bourne); 3,018,484 (Koehn); 3,263,682 (Rosenfield); 3,348,541 (Loebeck).

While the devices disclosed in the aforementioned discussion may be generally suitable for their intended purposes, they suffer from bulkiness and/or expense. Thus, there remains a need for a lightweight and easy-to-apply covering for a wound, scrape or abrasion on the end of an appendage. The present invention meets that need.

All references cited herein are incorporated herein by reference in their entireties.

### BRIEF SUMMARY OF THE INVENTION

A bandage for covering a wound on a tip of an appendage (e.g., a finger or toe) is disclosed. The bandage comprises: an appendage tip receptacle (ATR) configured for receipt of the appendage tip therein, the ATR comprising an enclosure having an inside surface that comprises an absorbent layer (e.g., antimicrobial gauze) thereon; and a pair of wings, one on each side of a lower region of the ATR, each of wings comprising an adhesive region (e.g., a hypoallergenic pressure sensitive adhesive, etc.) and wherein the pair of wings is configured for wrapping around a lower portion of the appendage for releasably securing the bandage to the appendage.

A method of forming a bandage for covering a wound on a tip of an appendage (e.g., a finger or toe) is disclosed. The method comprises: forming an absorbent layer on a first surface of a first portion of material and forming an absorbent layer on a first surface of a second portion of material; forming an appendage tip receptacle (ATR) by joining an upper region of the first portion of material to an upper region of the second portion of material along edges of the upper region of the first portion and of the upper region of the second portion, the first surfaces facing each other, wherein the ATR is configured for receipt of the appendage tip therein; forming a pair of wings on the first portion of material, one wing on each side of a lower region of the first portion of material; and providing an adhesive region (e.g., a hypoallergenic pressure sensitive adhesive, etc.) on each wing located on a side of each wing that is adjacent the second portion of material.

A method of securing a bandage over a wound on the tip of an appendage (e.g., a finger or toe) is disclosed. The method comprises: providing an absorbent layer (e.g., antimicrobial gauze) on a first surface of a first portion of material and providing an absorbent layer on a first surface of a second portion of material; forming a pair of wings on the first portion of material, one wing on each side of a lower region of the first portion of material and including an adhesive region (a hypoallergenic pressure sensitive adhesive, etc.) on each wing; each of the adhesive regions initially comprising a removable liner thereon; forming an appendage tip receptacle (ATR) by joining the first and second portions of material together along edges of upper regions of the first and second portions such that the first surfaces are facing each other to form an enclosure; inserting the appendage with the wound inside the ATR; removing the removable liners; and wrapping the wings in opposite directions around the appendage such that portions of the wings overlap and applying pressure to the overlapping portions to releasably secure the bandage to the appendage.

### BRIEF DESCRIPTION OF SEVERAL VIEWS OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 depicts the bandage of the present invention applied to the end of a finger;

FIG. 2 depicts the bandage of the present invention applied to the end of a toe;

FIG. 3 is a top front isometric view of the present invention before it is applied to an appendage;

FIG. 4 is a top-front isometric view of the present invention shown in its operative condition of covering the wound (appendage is omitted from view);

FIG. 5 is an exploded view of the present invention, depicting its construction;

FIG. 6 is a right-side view of the present invention during use, covering a wound on the appendage;

FIG. 7A is a view of the present invention taken along line 7A-7A of FIG. 4;

FIG. 7B is an enlarged partial cross-sectional view of portion of the present invention indicated in FIG. 7A; and

FIG. 8 is a view of the bandage of the present invention initially applied to the tip of the appendage (e.g., a finger shown by way of example only), with the WSA and just before the release liners are removed and the two wings are wrapped around the lower region of the second portion for releasably securing the bandage to the appendage.

### 1DETAILED DESCRIPTION OF THE PREFERRED EMBODIMENTS

Referring now to the figures, wherein like reference numerals represent like parts throughout the several views, exemplary embodiments of the present disclosure will be described in detail. Throughout this description, various components may be identified having specific values, these values are provided as exemplary embodiments and should not be limiting of various concepts of the present invention as many comparable sizes and/or values may be implemented.

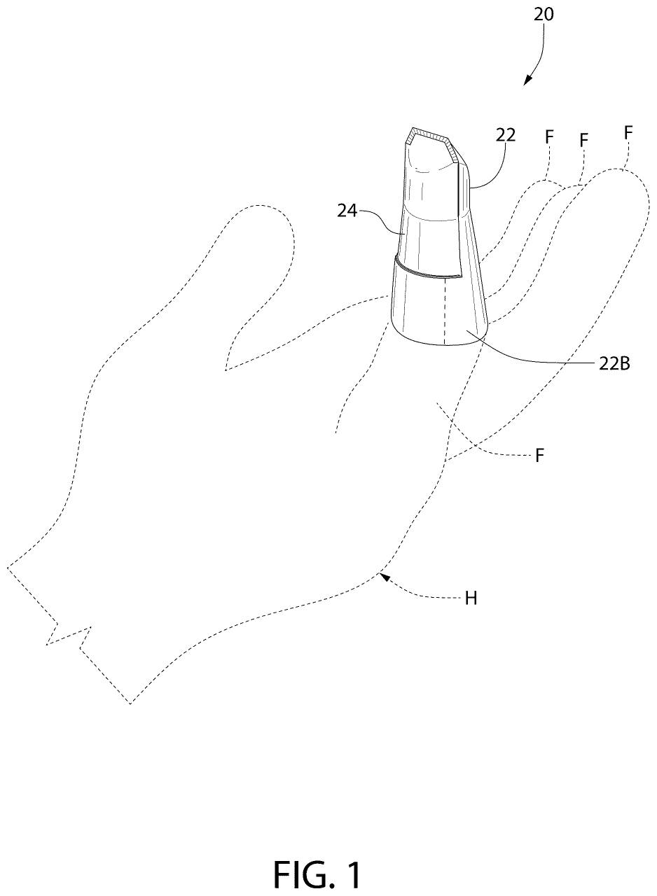

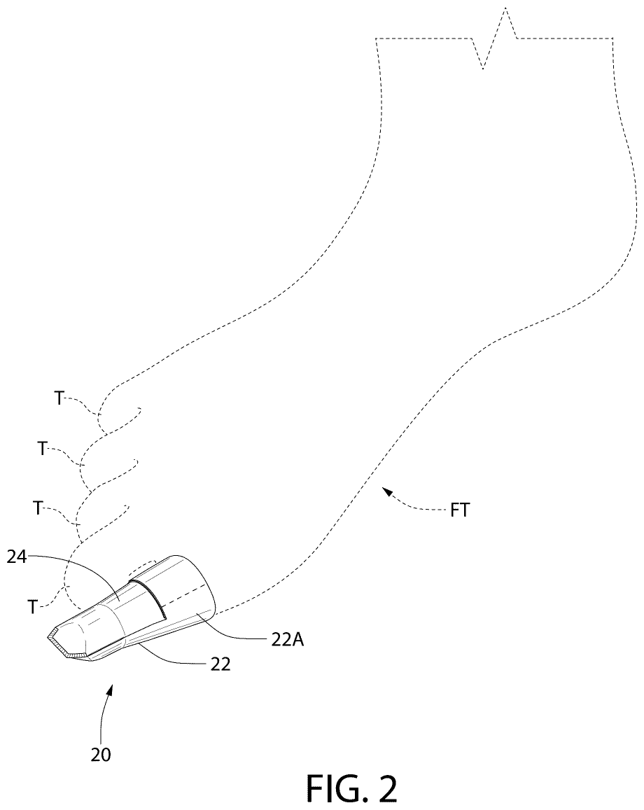

FIG. 1 depicts the appendage tip bandage (ATB) 20 applied to the tip of an appendage (e.g., a pinky finger) F of a hand H, showing it covering the entire tip of the finger F. FIG. 2 shows another ATB 20 but applied to a toe T of a foot FT, also showing it covering the entire tip of the toe T. As can be seen in these figures, the ATB 20 conforms to the forward end of the finger F and toe T, thereby fully covering a wound, scrape or abrasion (WSA, not shown) present on the tip of appendage of the hand H or foot F.

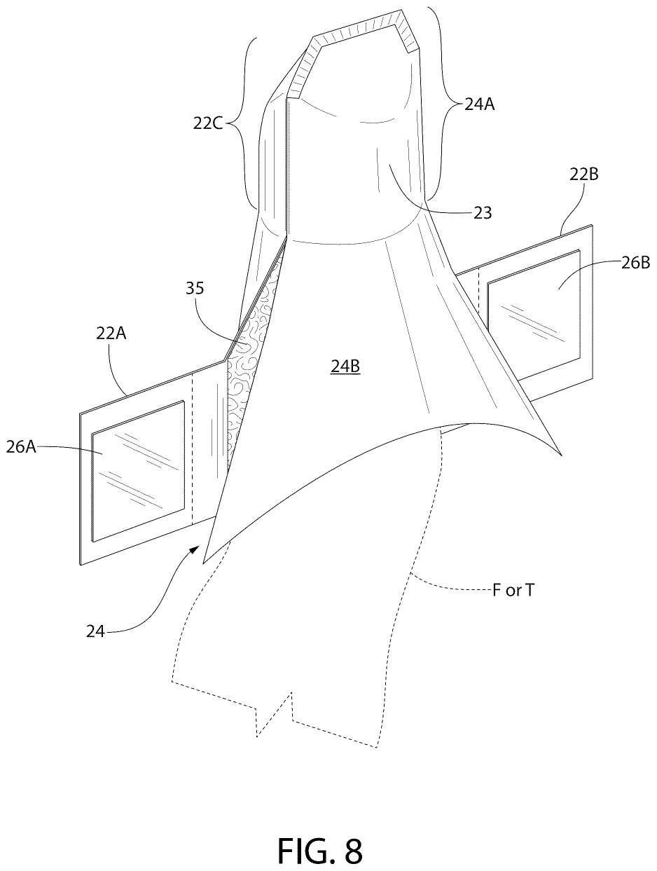

The ATB 20 comprises a first portion 22 having wings 22A and 22B and a second portion 24 that is fixedly coupled to the first portion 22. With the first portion 22 and the second portion 24 joined together, they define an “appendage tip receptacle” (ATR) 23 for receiving the appendage therein, as shown in FIG. 8, for completely covering the WSA. This ATR 23 forms an enclosure having an inside surface that is covered with an absorbent layer 32, as will be discussed in detail below.

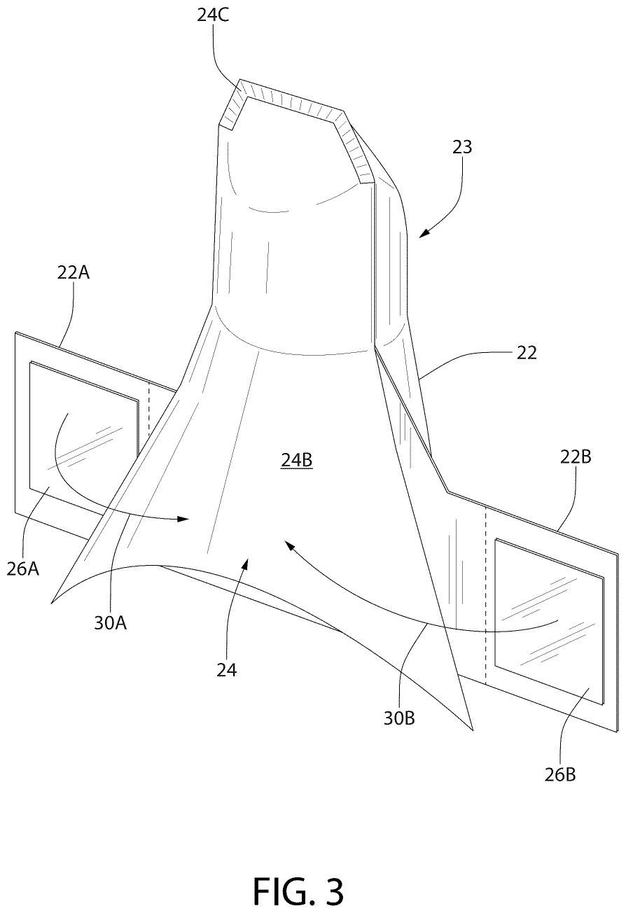

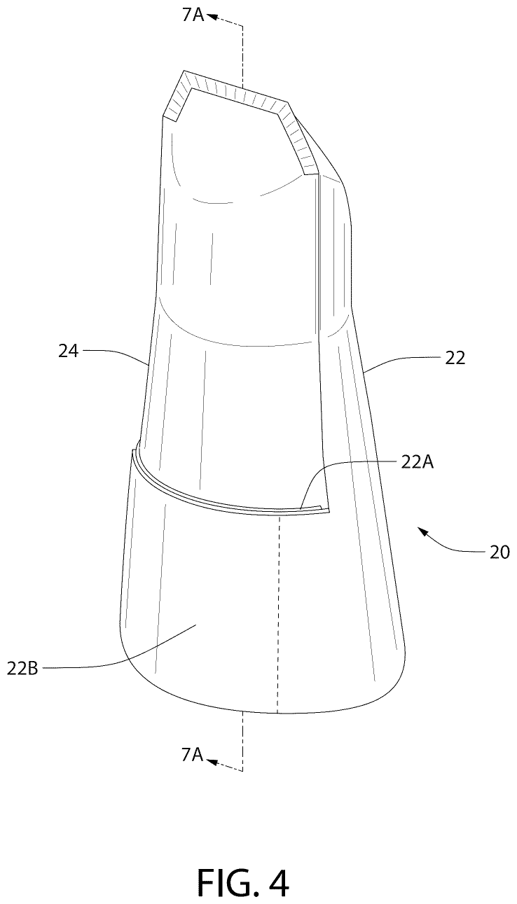

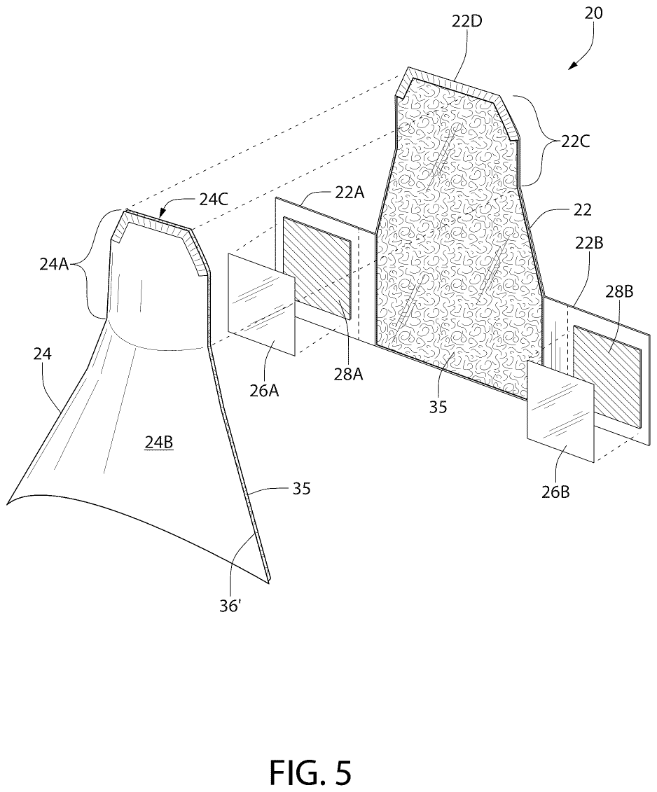

Once the appendage is positioned inside the receptacle, removable liners 26A and 26B are removed from the wings 22A and 22B respectively, which expose respective adhesive regions 28A and 28B (FIG. 5). These adhesive regions 28A and 28B are located on the surfaces of the wings 22A/22B that are adjacent the second portion 24. These adhesive regions may comprise various kinds of adhesives, including hypoallergenic pressure sensitive adhesives, etc. The user then wraps the wings 22A/22B in the respective directions 30A and 30B (FIG. 3) over a lower region 24B of the second portion 24, with the wings 22A/22B overlapping one another, thereby releasably securing the ATB 20 to the appendage, as shown in FIGS. 1-2. FIG. 4 depicts the ATB 20 in its operative condition with the appendage omitted for clarity. As can be seen in FIG. 4, wing 22B is overlapping wing 22A, it being understood that wing 22A could just as easily have overlapped wing 22B.

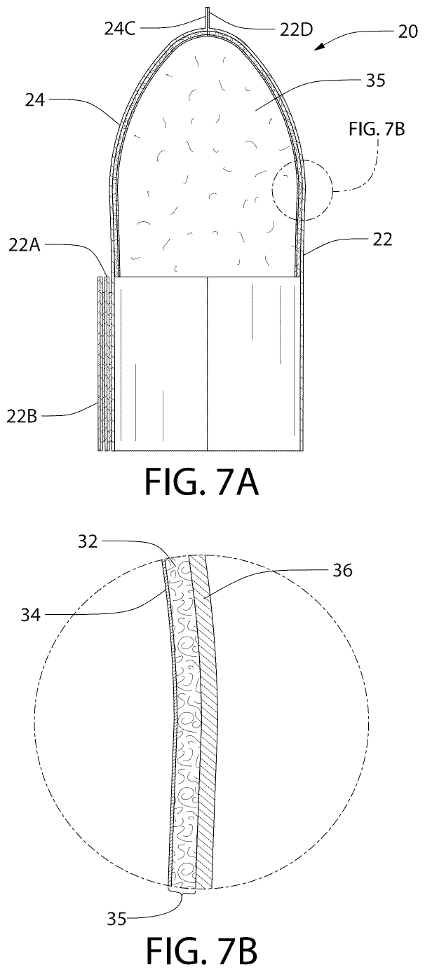

As can be seen most clearly in FIG. 5, the inside of the first portion 22 comprises a central portion having an absorbent element 32 (e.g., antimicrobial gauze). On top of this absorbent element 32 is a very thin polypropylene layer 34, which can be best be seen in FIG. 7B. The combination of the absorbent layer 32 and the thin polypropylene layer 34 is hereinafter referred to as the wound contact layer 35 (FIG. 7B). The outer layer 36 (FIG. 7B) of the first portion 22 comprises a flexible plastic (e.g., polyethylene) material.

Although not easily seen, the inner surface of the second portion 24 also comprises a wound layer 35 secured to an outer layer 36′ (FIG. 5) which may also comprise a flexible plastic (e.g., polyethylene) film. Thus, with the first portion 22 and the second portion 24 secured together the wound layers 35 provide a coverage of 360° on the tip of an appendage (e.g., finger F or toe T), as most clearly seen by the cross-sectional view of FIG. 7A.

These outer layers 36 and 36′ of flexible plastic are similar to the composition of conventional adhesive bandages.

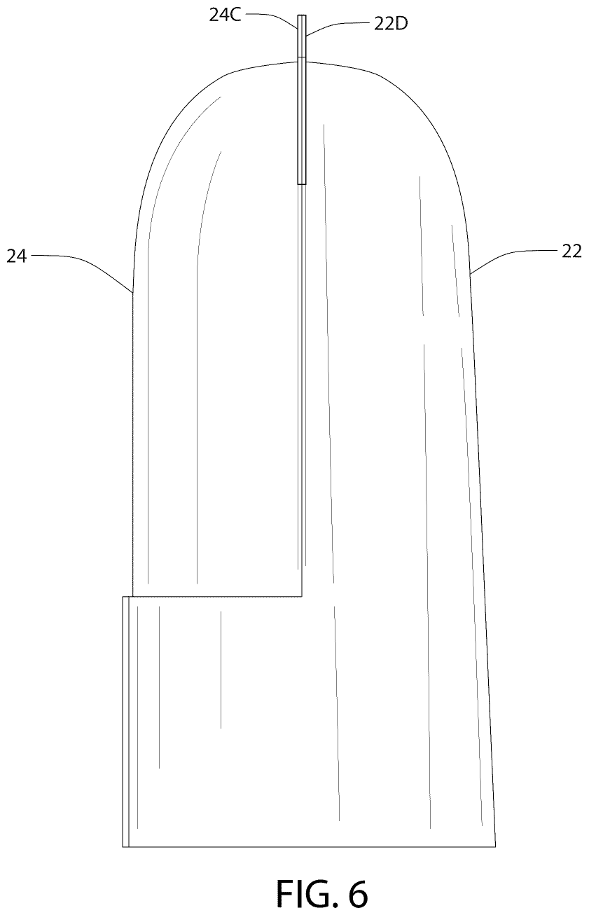

Although there are many ways of securing the first portion 22 and second portion 24 together, some exemplary methods are by heat bonding or ultrasonic bonding. As shown most clearly in FIG. 6, an upper region 22C of the first portion 22 is secured to an upper portion 24A of the second portion 24 along their edges with which they are in contact. The upper edge 22D and the upper edge 24C of the second portion 24 are contoured to provide a more “rounded” profile at the top of the ATB 20. Although corners do appear on these upper edges 22D/24C, it is within the broadest scope of the present invention that these edges also include a smooth circular contour.

With the first portion 22 and second portion 24 joined together the lower region 24C of the second portion 24 forms a “skirt” that is unattached. However, once the wings 22A/22B are wrapped around this lower region 24C, this lower region 24C is brought into close contact with the first portion 22.

To use the ATB 20, the user would remove the ATB 20 from a sterile pouch (not shown), leaving the ATB 20 prior to use, as shown in FIG. 3. Next, the user would insert the appendage (finger F or toe T) with the WSA into the receptacle portion of ATB 20. Next, the user would remove the liners 26A and 26B from the respective wings 22A/22B. At this point, the user would then wrap the wings 22A/22B in the directions 30A/30B (FIG. 3) to overlap one another. By doing this, as shown in FIGS. 1 and 2 the ATB 20 is firmly secured to the finger F or toe T having the WSA.

It should be noted that different sizes of the ATB 20 may be provided in one package to properly fit different finger sizes and different toe sizes. For example, adult-sized ATBs 20 may be provided in two to three different sizes while child-size ATBs 20 may be provided in two to three different smaller sizes.

### Reference Characters

F Finger;

FT foot;

H hand;

T toe

WSA (wound, scrape or abrasion);

20 appendage tip bandage (ATB);

22 first portion;

22A left wing;

22B right wing;

22C upper region of first portion;

22D upper edge of first portion;

23 appendage tip receptacle (ATR)

24 second portion;

24A upper region of second portion;

24B lower region of second portion;

24C upper edge of second portion

26A left release liner;

26B right release liner;

28A left adhesive region;

28B right adhesive region;

30A left wing wrap direction;

30B right wing wrap direction;

32 absorbent layer;

34 very thin polypropylene layer on absorbent layer;

35 wound layer comprising absorbent layer and very thin polypropylene layer

36 outer layer of first portion;

36′ Outer Layer of Second Portion.

While the invention has been described in detail and with reference to specific examples thereof, it will be apparent to one skilled in the art that various changes and modifications can be made therein without departing from the spirit and scope thereof.

What is claimed is:

1. A bandage for covering a wound on a tip of an appendage, said bandage comprising:
an appendage tip receptacle (ATR) configured for receipt of the appendage tip therein, said ATR comprising an enclosure having an inside surface that comprises an absorbent layer thereon; and
a pair of wings, one on each side of a lower region of said ATR, each of said wings comprising an adhesive region and wherein said pair of wings is configured for wrapping around a lower portion of the appendage for releasably securing said bandage to the appendage.

2. The bandage of claim 1 wherein said adhesive regions each initially comprises a removeable liner thereon.

3. The bandage of claim 1 wherein said ATR comprises:
a first portion having an upper region and said lower region;
a second portion having an upper region and a lower region; and
wherein said first portion and said second portion are secured together along edges of said upper region of said first portion and said upper region of said second portion to form said enclosure.

4. The bandage of claim 1 wherein a layer of polypropylene is applied to said absorbent layer.

5. The bandage of claim 1 wherein said absorbent layer comprises antimicrobial gauze.

6. The bandage of claim 1 wherein said first portion and said second portion comprise flexible plastic.

7. The bandage of claim 6 wherein said flexible plastic comprises polyethylene.

8. The bandage of claim 1 wherein said adhesive regions comprise a hypoallergenic pressure sensitive adhesive.

9. A method of forming a bandage for covering a wound on a tip of an appendage, said method comprising:
forming an absorbent layer on a first surface of a first portion of material and forming an absorbent layer on a first surface of a second portion of material;
forming an appendage tip receptacle (ATR) by joining an upper region of a first portion of material to an upper region of a second portion of material along edges of said upper region of said first portion and of said upper region of said second portion, said first surfaces facing each other, said ATR configured for receipt of the appendage tip therein;
forming a pair of wings on said first portion of material, one wing on each side of a lower region of said first portion of material; and
providing an adhesive region on each wing located on a side of each wing that is adjacent said second portion of material.

10. The method of claim 9 wherein said step of applying said absorbent layers comprises applying a layer of polypropylene to said absorbent layers.

11. The method of claim 9 wherein said absorbent layer is antimicrobial gauze.

12. The method of claim 9 wherein said material comprises flexible plastic.

13. The method of claim 12 wherein said flexible plastic comprises polyethylene.

14. The method of claim 9 wherein said adhesive regions comprise a hypoallergenic pressure sensitive adhesive.

15. The method of claim 9 further comprising the step of initially applying removable liners to said adhesive regions.

16. A method of securing a bandage over a wound on the tip of an appendage, said method comprises:
providing an absorbent layer on a first surface of a first portion of material and providing an absorbent layer on a first surface of a second portion of material;
forming a pair of wings on the first portion of material, one wing on each side of a lower region of said first portion of material and including an adhesive region on each wing; each of said adhesive regions initially comprising a removable liner thereon;
forming an appendage tip receptacle (ATR) by joining said first and second portions of material together along edges of upper regions of said first and second portions such that said first surfaces are facing each other to form an enclosure;
inserting the appendage with the wound inside said ATR;
removing said removable liners; and
wrapping said wings in opposite directions around the appendage such that portions of said wings overlap and applying pressure to said overlapping portions to releasably secure said bandage to the appendage.

17. The method of claim 16 wherein said absorbing layer comprises an antimicrobial gauze.

18. The method of claim 16 wherein said adhesive regions comprise a hypoallergenic pressure sensitive adhesive.

19. The method of claim 16 wherein said steps of providing said absorbent layers comprises applying a layer of polypropylene to said absorbent layers.

20. The method of claim 16 wherein said first and second portions of material comprise flexible plastic.
